(19) United States
(12) Patent Application Publication
(10) Pub. No.: US 2023/0172700 A1
(43) Pub. Date: Jun. 8, 2023

(54) A COLLAGEN-HYDROXYAPATITE DEVICE FOR NON-SURGICAL PERIODONTAL TREATMENT

(71) Applicant: DATUM DENTAL LTD., Lod (IL)

(72) Inventors: Yuval ZUBERY, Cochav Yair (IL); Arie GOLDLUST, Ness Ziona (IL); Thomas BAYER, Tel Aviv (IL)

(73) Assignee: DATUM DENTAL LTD., Lod (IL)

(21) Appl. No.: 17/781,433

(22) PCT Filed: Dec. 4, 2021
(86) PCT No.: PCT/IB2019/060449

§ 371 Date: Jun. 1, 2022

(51) Int. Cl. A61C 19/06; A61K 6/54; A61K 6/52; A61K 6/69; A61K 6/75

(52) U.S. Cl. CPC A61C 19/063; A61K 6/52; A61K 6/54; A61K 6/69; A61K 6/75

(57) ABSTRACT

This invention provides a non-surgical method for treating periodontitis comprising administering an article into a periodontal pocket, where the article comprises crosslinked collagen and hydroxyapatite. This invention also provides articles to be used within these methods.

### FIELD OF THE INVENTION

This invention provides a non-surgical method for treating periodontitis comprising administering an article into a periodontal pocket, where the article comprises crosslinked collagen and hydroxyapatite. This invention also provides articles to be used within these methods.

### BACKGROUND OF THE INVENTION

Periodontitis is a disease where the periodontal tissue is chronically infected, continuously receding and exposing the root surface to the oral environment, frequently forming a pocket that might be infected. This can cause loosening and finally loss of the tooth and its supporting bone. Periodontal treatment is often aimed at debridement of the root surface and attempting to decrease the pocket depth via reattachment to the root surface or reduction of the non-attached gingival tissue.

Periodontal treatment usually begins with non-surgical debridement (scaling and root planning) followed by meticulous maintenance (both professional and patient’s own oral hygiene measures). When this is not sufficient and further attachment loss is detected, resective or regenerative surgical procedures are employed in an attempt to stop the disease progression and even achieve regeneration of lost periodontal tissues.

Accordingly, there is an unmet need for alternative, minimally invasive, safer, less pain-causing and easy-to-perform methods of treating periodontitis.

This invention provides a non-surgical method for treating periodontitis comprising administering an article into a periodontal pocket, where the article comprises crosslinked collagen and hydroxyapatite.

### SUMMARY OF THE INVENTION

In one embodiment, this invention provides a non-surgical method for treating periodontitis comprising administering an article into a periodontal pocket, where the article comprises crosslinked collagen and hydroxyapatite. In another embodiment, the method leads to reduction of the periodontal pocket depth.

### BRIEF DESCRIPTION OF THE DRAWINGS

The subject matter regarded as the invention is particularly pointed out and distinctly claimed in the concluding portion of the specification. The invention, however, both as to organization and method of operation, together with objects, features, and advantages thereof, may best be understood by reference to the following detailed description when read with the accompanying drawings in which:

FIG. 1 depicts shapes of the article used in this invention.

FIGS. 2A-2B depict histological analysis microphotographs of 5 mm defect of animal 1 that was overfilled with crosslinked collagen-hydroxyapatite article and covered with OSSIX® PLUS, where: “I” is inflammation, “NB” is new bone, “OB” is original bone, “OP” is Ossix® Plus and “OPB” is the crosslinked collagen-hydroxyapatite article. FIG. 2A: double magnification; and FIG. 2B: five times magnification and focus on the interface region between the crosslinked collagen-hydroxyapatite article and a newly formed bone .

FIGS. 3A-3B depict X-ray analysis of animals 1 and 2 within the bone grafting study. FIG. 3A: animal 1; and FIG. 3B: animal 2.

FIGS. 4A-4B depict histological analysis microphotographs of 10 mm defect of animal 2 that was overfilled with crosslinked collagen-hydroxyapatite article and covered with OSSIX® PLUS, where: “F” is fibroblasts, “NB” is new bone, “OB” is original bone, “OP” is Ossix® Plus and “OPB” is the crosslinked collagen-hydroxyapatite article. FIG. 4A: double magnification; and FIG. 4B: five times magnification and focus on the region comprising a newly formed bone; the residual defect; and the crosslinked collagen-hydroxyapatite article.

It will be appreciated that for simplicity and clarity of illustration, elements shown in the Figures have not necessarily been drawn to scale. For example, the dimensions of some of the elements may be exaggerated relative to other elements for clarity. Further, where considered appropriate, reference numerals may be repeated among the Figures to indicate corresponding or analogous elements.

### DETAILED DESCRIPTION OF THE PRESENT INVENTION

In the following detailed description, numerous specific details are set forth in order to provide a thorough understanding of the invention. However, it will be understood by those skilled in the art that the present invention may be practiced without these specific details. In other instances, well-known methods, procedures, and components have not been described in detail so as not to obscure the present invention.

### Non-Surgical Methods for Treating Periodontitis

In one aspect, this invention provides a non-surgical method for treating periodontitis comprising administering an article into a periodontal pocket, wherein the article comprises crosslinked collagen and hydroxyapatite. In one embodiment, the periodontal pocket is infected or not infected. In another embodiment, the periodontal pocket is infected. In another embodiment, the infection is bacterial or microbial infection. In another embodiment, the periodontal pocket is not infected. Each possibility represents a separate embodiment of the invention.

In one embodiment, the method of this invention leads to reduction of the periodontal pocket depth. Without being bound by any mechanism or theory, it is contemplated that the collagen-hydroxyapatite article stimulates bone and soft tissue growth in the pocket region, leading to “closing” of the pocket and reduction of depth thereof. It is further contemplated that the collagen (within the article) serves as a substrate for a collagenase enzyme in the pocket and as such it can saturate the active site of the enzyme, inhibiting degradation of the gingival tissue; once applied in the periodontal pocket, the article is absorbed in the gingival crevicular fluid, and is turned into a hydrated soft scaffold that slows the collagenases mediated destruction of the gingival tissue by absorbing the enzyme and saturating its active site. It is further contemplated that the hydroxyapatite (within the article) serves as a solid resin for absorbing and immobilizing of the collagenase enzyme..

In one embodiment, the method of this invention does not include administration of an additional pharmaceutically active agent.

In one additional embodiment, the method of this invention further comprises administration of at least one pharmaceutically active agent selected from the group consisting of: antibacterial agents, antifungal agents, antiseptic agents, anti-inflammatory agents, antibiotic agents, vitamins and vitamers and any combination thereof. In another embodiment, the pharmaceutically active agent is an antibacterial agent. In another embodiment, the pharmaceutically active agent is an antifungal agent. In another embodiment, the pharmaceutically active agent is an antiseptic agent. In another embodiment, the pharmaceutically active agent is an anti-inflammatory agent. In another embodiment, the pharmaceutically active agent is an antibiotic agent. In another embodiment, the pharmaceutically active agent is a vitamin and/or a vitamer. Each possibility represents a separate embodiment of the invention.

In another embodiment, non-limiting examples of antibacterial agents include: Amikacin, Gentamicin, Kanamycin, Neomycin, Netilmicin, Tobramycin, Paromomycin, Arbekacin, Plazomicin, Streptomycin, Apramycin , Geldanamycin, Herbimycin, Loracarbef, Faropenem, Ertapenem, Doripenem, Imipenem, Meropenem, Cefazolin, Cefacetrile, Cefadroxil, Cephalexin, Cefaloglycin, Cefalonium, Cefaloridine, Cefalotin, Cefapirin, Cefatrizine, Cefazedone, Cefazaflur, Cefradine, Cefroxadine, Ceftezole, Cefaclor, Cefamandole, Cefminox, Cefonicid, Ceforanide, Cefotiam, Cefprozil, Cefbuperazone, Cefuroxime, Cefuzonam, Cephamycin, Cefoxitin, Cefotetan, Cefmetazole, Carbacephem, Cefixime, Ceftazidime, Ceftriaxone, Cefcapene, Cefdaloxime, Cefdinir, Cefditoren, Cefetamet, Cefmenoxime, Cefodizime, Cefoperazone, Cefotaxime, Cefpimizole, Cefpiramide, Cefpodoxime, Cefsulodin, Cefteram, Ceftibuten, Ceftiolene, Ceftizoxime, Oxacephem, Cefepime, Cefozopran, Cefpirome, Cefquinome, Ceftiofur, Cefquinome, Cefovecin, CXA-101, Ceftaroline, Ceftobiprole, Clindamycin, Lincomycin, Azithromycin, Clarithromycin, Dirithromycin, Erythromycin, Roxithromycin, Troleandomycin, Telithromycin, Spectinomycin, Solithromycin, Aztreonam, Furazolidone, Nitrofurantoin, Amoxicillin, Ampicillin, Azlocillin, Carbenicillin, Cloxacillin, Dicloxacillin, Flucloxacillin, Mezlocillin, Methicillin, Nafcillin, Oxacillin, Penicillin G, Penicillin V, Piperacillin, Temocillin, Ticarcillin, iprofloxacin, Enoxacin, Gatifloxacin, Levofloxacin, Lomefloxacin, Moxifloxacin, Nalidixic acid, Levonadifloxacin, Norfloxacin, Ofloxacin, Trovafloxacin, Grepafloxacin, Sparfloxacin, Temafloxacin, Delafloxacin, Mafenide, Sulfonamidochrysoidine, Sulfacetamide, Sulfadiazine, Sulfamethizole, Sulfamethoxazole, Sulfasalazine, Sulfisoxazole, Trimethoprim, emeclocycline, Doxycycline, Minocycline, Oxytetracycline, Tetracycline, Tigecycline, Tedizolid, Linezolid, Ranbezolid, Torezolid, Radezoli, any combination thereof and any pharmaceutically acceptable salt thereof. Each possibility represents a separate embodiment of the invention.

In another embodiment, non-limiting examples of antifungal agents include: terbinafine, naftifine, amphotericin B, butenafine, chloroxylenol, ciclopirox, flucytosine, caspofungin, griseofulvin, clotrimazole, fluconazole, itraconazole, ketoconazole, miconazole, oxiconazole , nystatin, undecylenic acid, any combination thereof and any pharmaceutically acceptable salt thereof. Each possibility represents a separate embodiment of the invention.

In another embodiment, non-limiting examples of anti-septic agents include: naftifine, tolnaftate, mediocidin, candicidin, trichomycin, hamycin, aurefungin, ascosin, ayfattin, azacolutin, trichomycin, levorin, heptamycin, candimycin, griseofulvin, pradimicins, benanomicin; ambisome; nikkomycin Z; flucytosine, perimycin, any combination thereof and any pharmaceutically acceptable salt thereof. Each possibility represents a separate embodiment of the invention.

In another embodiment, non-limiting examples of anti-inflammatory agents include: aspirin, ibuprofen, naproxen, celecoxib, diclofenac, ketoprofen, ketorolac, oxaprozin, salsalate, sulindac, any combination thereof and any pharmaceutically acceptable salt thereof. Each possibility represents a separate embodiment of the invention.

In another embodiment, non-limiting examples of antibiotics include penicillin, cephalosporin, ciprofloxacin, erythromycin, any combination thereof and any pharmaceutically acceptable salt thereof. Each possibility represents a separate embodiment of the invention.

In another embodiment, non-limiting examples of vitamins and vitamers include: vitamin a, retinol, retinal, carotenoid, vitamin B1, thiamine, vitamin B2, riboflavin, vitamin B3, niacin, niacinamide, nicotinamide, riboside, vitamin B5, pantothenic acid, vitamin B6, pyridoxine, pyridoxamine, pyridoxal, vitamin B7, biotin, vitamin B9, folates, vitamin B12, cyanocobalamin, hydroxocobalamin, methylcobalamin, adenosylcobalamin, vitamin C, ascorbic acid, vitamin D, cholecalciferol (D3), ergocalciferol (D2), vitamin E, tocopherols, tocotrienols, vitamin K, phylloquinone, menaquinones, any combination thereof and any pharmaceutically acceptable salt thereof. Each possibility represents a separate embodiment of the invention.

In one embodiment, article administration into a periodontal pocket comprises insertion or pushing of the article into the pocket. In another embodiment, the insertion or pushing is performed to allow at least partial contact between the article and the pocket. In another embodiment, the insertion or pushing is performed to allow full contact between the article and the pocket.

In one embodiment, the method of this invention further comprises scaling and root planning, where the scaling and root planning is performed prior to article’s administration. In another embodiment, scaling and root planning is performed in any method as known in the art of this invention.

In one embodiment, the method of this invention further comprises covering the article of this invention with a crosslinked collagen membrane (e.g. Ossix® Plus), thus the article contacts (e.g. inserted within) the periodontal pocket and the collagen membrane is found on top of it. In another embodiment, fixation sutures are placed over the collagen membrane.

### Articles Used Within Methods of This Invention

In one further aspect, this invention provides an article comprising crosslinked collagen and hydroxyapatite for use in the methods as described hereinabove. In one embodiment, the article is biodegradable and biocompatible.

In some embodiments, the weight ratio of collagen to hydroxyapatite within the article used in this invention is between 95:5 and 20:80. In another embodiment, the ratio is between 90:10 and 25:75. In another embodiment, the ratio is between 85:15 and 30:70. In another embodiment, the ratio is between 80:20 and 35:65. In another embodiment, the ratio is between 75:25 and 40:60. In another embodiment, the ratio is between 70:30 and 45:55. In another embodiment, the ratio is 70:30. Each possibility represents a separate embodiment of the invention.

In some embodiments, the concentration of cross-linked collagen within the article used in this invention is between 20 to 95 w/w. In another embodiment, the concentration is between 20 to 30 w/w. In another embodiment, the concentration is between 30 to 40 w/w. In another embodiment, the concentration is between 40 to 50 w/w. In another embodiment, the concentration is between 50 to 60 w/w. In another embodiment, the concentration is between 60 to 70 w/w. In another embodiment, the concentration is between 70 to 80 w/w. In another embodiment, the concentration is between 80 to 90 w/w. In another embodiment, the concentration is between 90 to 95 w/w. Each possibility represents a separate embodiment of the invention

In some embodiments, the concentration of hydroxyapatite within the article used in this invention is between 5 to 80 w/w. In another embodiment, the concentration is between 5 to 10 w/w. In another embodiment, the concentration is between 10 to 20 w/w. In another embodiment, the concentration is between 20 to 30 w/w. In another embodiment, the concentration is between 30 to 40 w/w. In another embodiment, the concentration is between 40 to 50 w/w. In another embodiment, the concentration is between 50 to 60 w/w. In another embodiment, the concentration is between 60 to 70 w/w. In another embodiment, the concentration is between 70 to 80 w/w. Each possibility represents a separate embodiment of the invention

In some embodiments, the article used in this invention further comprises one pharmaceutically active agent as described hereinabove.

In some embodiments, the pharmaceutically active agent as described hereinabove can be administered in addition and/or within the administered article which comprises crosslinked collagen and hydroxyapatite. In one embodiment, when the method of this invention further comprises administration of a pharmaceutically active agent, the administered article comprises or does not comprise a pharmaceutically active agent as described hereinabove, in addition to the crosslinked collagen and the hydroxyapatite. In one embodiment, when the method of this invention does not comprise administration of a pharmaceutically active agent, the administered article comprises or does not comprise a pharmaceutically active agent as described hereinabove, in addition to the crosslinked collagen and the hydroxyapatite. Each possibility represents a separate embodiment of the invention

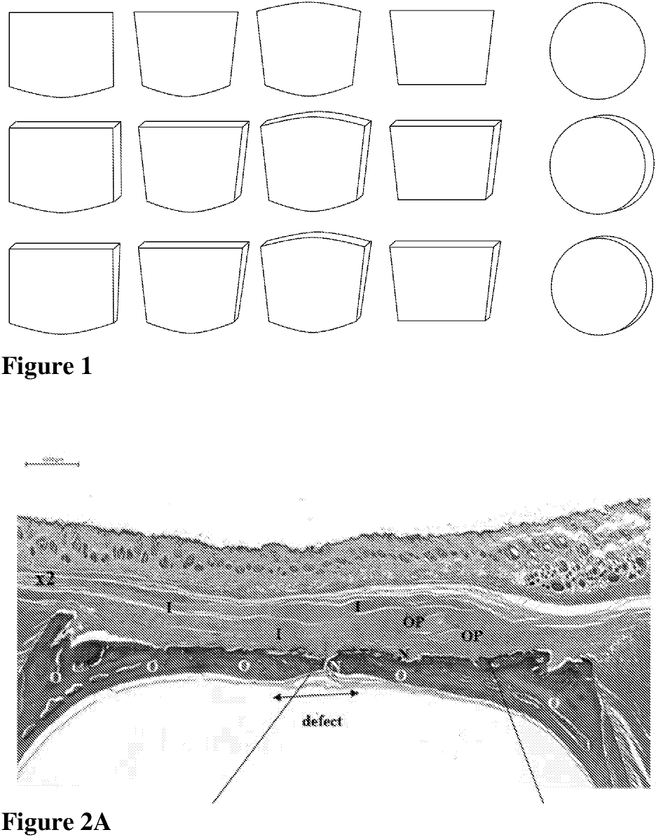

In some embodiments, the term “article” as used herein refers to a matrix which is provided in some shape or form, i.e. compressed, designed, engineered, manufactured or prepared as known in the art to provide some two or three-dimensional structure of such article. In other embodiments, non-limiting examples of the two or three-dimensional structures are depicted in FIG. 1. In other embodiments, any possible design, engineering, manufacturing or preparation process can be applied in order to provide the article. In another embodiment, the matrix is designed, compressed and/or shaped to provide an article as described hereinabove and fits into a periodonatal pocket. In another embodiment, the article is compressed in a force of between 0.005 and 10 ton-force. In another embodiment, the article is compressed in a force of between 0.1 and 6 ton-force. In another embodiment, the article is compressed in a force of between 0.0055 and 0.2 ton-force. In another embodiment, the article is compressed in a force of between 0.005 and 0.02 ton-force. In another embodiment, the article is compressed in a force of between 0.02 and 0.05 ton-force. In another embodiment, the article is compressed in a force of between 0.05 and 0.1 ton-force. In another embodiment, the article is compressed in a force of between 0.1 and 0.2 ton-force. In another embodiment, the article is compressed in a force of between 0.2 and 1 ton-force. In another embodiment, the article is compressed in a force of between 1 and 2 ton-force. In another embodiment, the article is compressed in a force of between 2 and 5 ton-force. In another embodiment, the article is compressed in a force of between 5 and 10 ton-force. Each possibility represents a separate embodiment of the invention.

In some embodiments, the term “matrix” as used herein refers to some physical extent of a solid matter (e.g. crosslinked collagen and hydroxyapatite). In other embodiments, the solid matter is dry and does not comprise any solvent or liquid. In other embodiments, the solid matter comprises at least one sole component. In another embodiment, non-limiting examples of a component include: a chemical compound, small molecule, metal, alloy, composite material, biomaterial, polymer and organometallic complex. In another embodiment, the solid matter comprises more than one component as selected from the foregoing list. In another embodiment, the matrix comprises a biocompatible material or composition. In another embodiment, the matrix is a biopolymer or a protein. In other embodiments, the physical attributes of the matrix afford its design, molding, carving out or engineering as known in the art into a desired shape. In another embodiment, non-limiting examples of attributes of the matrix include the following characteristics: dense, porous or non-porous, viscous, rigid, soft or moldable. In another embodiments, the carving out is done by any method as known in the art. In another embodiment, the carving out of the method of the invention is done by a CNC (Computer numerical control) machine, a laser cutting machine, a waterjet cutter, a driller or an abrasive device. Each possibility represents a separate embodiment of the invention. In another embodiment, the matrix has a porosity of between 10-90%. In another embodiment, the matrix has a porosity of between 10-20%. In another embodiment, the matrix has a porosity of between 20-30%. In another embodiment, the matrix has a porosity of between 30-40%. In another embodiment, the matrix has a porosity of between 40-50%. In another embodiment, the matrix has a porosity of between 50-60%. In another embodiment, the matrix has a porosity of between 60-70%. In another embodiment, the matrix has a porosity of between 70-80%. In another embodiment, the matrix has a porosity of between 80-90%. In another embodiment, the matrix has a porosity of between 10-30%. In another embodiment, the matrix has a porosity of between 30-50%. In another embodiment, the matrix has a porosity of between 50-70%. In another embodiment, the matrix has a porosity of between 70-90%. Each possibility represents a separate embodiment of the invention.

In some embodiments, the term “collagen” refers to a biopolymer organized in a fibrillar networks or other non-fibrillar superstructures, and it is the main component of connective tissue within the human or numerous animals’ body. Numerous types of collagen are known and found naturally. Non-limiting examples include types I-V. In some embodiments, collagen has a fibrillar (such as Type I) or non-fibrillar structure. Each possibility represents a separate embodiment of the invention.

In one embodiment, the collagen used in this invention refer to native collagen, fibrillar collagen, fibrillar atelopeptide collagen, lyophilized collagen, collagen obtained from animal sources, human collagen, recombinant collagen, pepsinized collagen, reconstituted collagen and any combination thereof. In another embodiment, the collagen includes fibrillar collagen reconstituted from monomolecular atelopeptide collagen. In another embodiment, the collagen is atelopeptide fibrillar collagen obtained by reconstituting monomolecular atelopeptide collagen obtained by proteolytic digestion of native collagen. Each possibility represents a separate embodiment of the invention.

In some embodiments, the term “cross-linked collagen” refers to a covalent network comprising biopolymer chains of collagen connected covalently and inter-molecularly with crosslinkers.

In some embodiments, the term “crosslinkers” refers to small molecules or polymers comprising at least two ends that can covalently connect polymeric/oligomeric chains and thereby crosslink these chains.

In some embodiments the collagen is cross-linked by a sugar. In another embodiment, the sugar is selected from the group consisting of glycerose (glyceraldehyde), threose, erythrose, lyxose, xylose, arabinose, ribose, allose, altrose, glucose, mannose, gulose, idose, galactose, talose and any combination thereof. In another embodiment, the sugar is glycerose (glyceraldehyde). Each possibility represents a separate embodiment of the invention.

In another embodiment, the sugar is a disaccharide. Each possibility represents a separate embodiment of the invention.

In another embodiment, the disaccharide is selected from the group consisting of maltose, lactose, sucrose, cellobiose, gentiobiose, melibiose, turanose, trehalose and any combination thereof. Each possibility represents a separate embodiment of the invention.

In another embodiment, the crosslinker can be any crosslinking agent as known in the art. In another embodiment, the crosslinker is a sugar. In another embodiment, the sugar is a compound represented by at least one of the following formulae I or II:

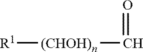

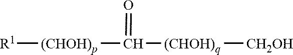

wherein:
R¹ is H or alkyl or alkenyl, an amino acid moiety, a peptide moiety, a saccharide moiety, a purine or a pyrimidine moiety, a phosphorylated purine or pyrimidine moiety;
n is an integer between 2-9, and
p and q are each independently an integer between 0-8, and the sum of p and q is at least 2 and not more than 8.

In another embodiment, the term “alkyl” group refers to a saturated aliphatic hydrocarbon, including straight-chain or branched-chain. In one embodiment, alkyl group is linear or branched. In another embodiment, alkyl is optionally substituted linear or branched. In one embodiment, the alkyl group has between 1-20 carbons. In one embodiment, the alkyl group has between 1-10 carbons. In one embodiment, the alkyl group has between 2-10 carbons. In one embodiment, the alkyl group has between 1-6 carbons. In one embodiment, the alkyl group has between 2-8 carbons. In another embodiment, non-limiting examples of alkyl groups include methyl, ethyl, propyl, isopropyl, isobutyl, butyl, pentyl, 3-pentyl, hexyl heptyl, octyl and hexadecyl. In another embodiment, the alkyl group is optionally substituted by one or more halogens, hydroxides, alkoxides, carboxylic acids, phosphates, phosphonates, sulfates, sulfonates amidates, cyanates, and a nitro group. Each possibility represents a separate embodiment of the invention.

In another embodiment, the term “alkenyl” group refers to an alkyl groups as described herein, having at least one carbon carbon double bond, including straight-chain and branched-chain groups. In one embodiment, the alkene has one double bond. In another embodiment, the alkene has more than one double bond. In another embodiment, the alkene has between 2-6 double bonds, each possibility represents a separate embodiment of this invention. In one embodiment, the alkene has 2-20 carbons. Non-limiting examples include ethylenyl, propylenyl, 2-methylpropyl-1-enyl and butenyl, each possibility represents a separate embodiment of this invention.

In another embodiment, the term “amino acid” refers to an organic compound containing amine (—NH2) and carboxyl (—COOH) functional groups, along with a side chain specific to each amino acid.. In another embodiment, any amino acid as known in the art can be utilized. In another embodiment, amino acid is alanine, arginine , asparagine, aspartic acid, cysteine, glutamine, glutamic acid, glycine, histidine, isoleucine, leucine, lysine, methionine, phenylalanine, proline, serine, threonine, tryptophan, tyrosine or valine.

In another embodiment, the term “peptide” refers to short chains of amino acids linked covalently via amide (peptide; —C(O)—N(H)—) bonds. In another embodiment, a peptide comprises between 2-20 amino acids. In another embodiment, a peptide is a dipepetide. In another embodiment, a peptide is a tripepetide. In another embodiment, a peptide is a tetrapepetide. In another embodiment, a peptide is a pentapepetide. In another embodiment, a peptide is a hexapepetide.

In another embodiment, the term “saccharide” refers to the group comprising sugars as described herein, cellulose and starch.

In another embodiment, the term “purine” refers to a heterocyclic aromatic organic compound that consists of a pyrimidine ring fused to an imidazole ring. Non limiting examples of purines include: purine, adenine, guanine, hypoxanthine, xanthine, theobromine, caffeine, uric acid and isoguanine.

In another embodiment, the term “pyrimidine” refers to a heterocyclic aromatic organic compound similar to pyridine but has an additional nitrogen within the aromatic ring so nitrogens are found in position 1, 3 of the ring. Non-limiting examples of purines include: cytosine, thymine and uracil.

In another embodiment, the term “a phosphorylated purine or pyrimidine” refers to a purine or pyrimidine as described herein, wherein the purine or pyrimidine is connected to a phosphoryl group (the chemical entity PO₃^{x}⁻; “x” denoting any possible protonation state).

In another embodiment, the sugar is a naturally occurring reducing sugar.

In another embodiment, the sugar is a diose, a triose, a tetrose, a pentose, a hexose, a septose, an octose, a nanose, or a decose. Each possibility represents a separate embodiment of the invention.

In another embodiment, the sugar is selected from the group consisting of glycerose (glyceraldehyde), threose, erythrose, lyxose, xylose, arabinose, ribose, allose, altrose, glucose, mannose, gulose, idose, galactose, talose and any combination thereof. Each possibility represents a separate embodiment of the invention.

In another embodiment, the sugar is a disaccharide. Each possibility represents a separate embodiment of the invention.

In another embodiment, the disaccharide is selected from the group consisting of maltose, lactose, sucrose, cellobiose, gentiobiose, melibiose, turanose, trehalose and any combination thereof. Each possibility represents a separate embodiment of the invention.

In some embodiments, the term “hydroxyapatite” refers to the naturally occurring calcium mineral with the formula of Ca₅(PO₄)₃(OH) or Caio(PO₄)₆(OH)₂. In other embodiments, hydroxyapatite can be prepared or obtained via any known method in the art. In another embodiment, bulk or nanoparticulate hydroxyapatite is utilized and/or prepared. Each possibility represents a separate embodiment of the invention.

In some embodiments, the term “nanoparticulate matter” (e.g. hydroxyapatite) refers to a matter which has at least one physical nanometric dimension. In another embodiment, nanoparticulate matter is shaped as nanoparticles, nanospheres, nanocubes, nanoplates, nanoribbons, nanowires, nanorods or in any other nanometric shape as known in the art.

### Method of Preparing Articles of This Invention

In one further aspect, this invention provides a method of preparing an article for use in a non-surgical method for treating periodontitis where the article comprises a cross-linked collagen and hydroxyapatite, and where the method comprises:
a. providing a matrix comprising cross-linked collagen and hydroxyapatite; and
b. shaping the matrix, thereby obtaining an article comprising a cross-linked collagen and hydroxyapatite.

In one embodiment, this invention provides a method of preparing a matrix comprising cross-linked collagen and hydroxyapatite, where the method comprises:
(i) mixing collagen and hydroxyapatite in a solution while collagen is fibrillated;
(ii) crosslinking of the collagen with a crosslinker, thereby obtaining a crosslinked slurry;
(iii) concentrating the crosslinked slurry, thereby obtaining a concentrated slurry;
(iv) lyophilizing the concentrated slurry, thereby obtaining a dry cake; and
(v) milling the dry cake, thereby obtaining a matrix comprising cross-linked collagen and hydroxyapatite.

In one embodiment, this invention provides a method of preparing a matrix comprising cross-linked collagen and hydroxyapatite, where the method comprises:
(i) providing an acidic solution of collagen, followed by neutralization of the solution, wherein the neutralization solution comprises hydroxyapatite;
(ii) concentrating the solution of step (i);
(iii)lyophilizing the concentrated mixture of step (ii), thereby obtaining a dried collagen composition;
(iv)incubating the composition with a crosslinker and a first solvent;
(v) washing the incubated composition of step (iv) with a second solvent; and
(vi)lyophilizing the washed composition of step (v), thereby obtaining a matrix comprising cross-linked collagen and hydroxyapatite.

In one embodiment, this invention provides a method of preparing a matrix comprising cross-linked collagen, hydroxyapatite and a pharmaceutically active agent where the method comprises:
(i) providing an acidic solution of collagen, followed by neutralization of the solution wherein the neutralization solution comprises hydroxyapatite;
(ii) concentrating the solution of step (i);
(iii)lyophilizing the concentrated mixture of step (ii), thereby obtaining a dried collagen composition;
(iv)incubating the composition with a crosslinker, a first solvent and adding a pharmaceutically active agent;
(v) washing the incubated composition of step (iv) with a second solvent; and
(vi)lyophilizing the washed composition of step (v), thereby obtaining a matrix comprising cross-linked collagen, hydroxyapatite and a pharmaceutically active agent.

In some embodiments, compressing steps (applying mechanical pressure using a specialized equipment) are applied in addition to, or instead of the lyophilization steps.

### Method of Preparing an Article of This Invention Using a Mold

In one additional aspect, this invention provides a method of preparing an article comprising a cross-linked collagen, hydroxyapatite, and the method comprises:
(i) providing an acidic solution of collagen, followed by neutralization of the solution, wherein the neutralization solution comprises hydroxyapatite;
(ii) concentrating the solution of step (i); and pouring it into a mold with a pre designed matrix shape;
(iii)lyophilizing the concentrated mixture of step (ii), thereby obtaining a dried composition comprising collagen and hydroxyapatite;
(iv)incubating the composition with a crosslinker and a first solvent;
(v) washing the incubated composition of step (iv) with a second solvent; and
(vi)lyophilizing the washed composition of step (v), thereby obtaining an article comprising a cross-linked collagen and hydroxyapatite.

In one embodiment, this invention provides a method of preparing an article comprising across-linked collagen, hydroxyapatite and a pharmaceutically active agent, and the method comprises:
(i) providing an acidic solution of collagen and a crosslinker, followed by neutralization of the solution, wherein the neutralization solution comprises hydroxyapatite;
(ii) concentrating the solution of step (i); and pouring it into a mold with a pre designed matrix shape;
(iii)lyophilizing the concentrated mixture of step (ii), thereby obtaining a dried composition comprising collagen and hydroxyapatite;
(iv)incubating the composition with the crosslinker, a first solvent and a pharmaceutically active agent;
(v) washing the incubated composition of step (iv) with a second solvent; and
(vi)lyophilizing the washed composition of step (v), thereby obtaining an article comprising a cross-linked collagen, hydroxyapatite and a pharmaceutically active agent.

In some embodiments, compressing steps (applying mechanical pressure using a specialized equipment) are applied in addition to, or instead of the lyophilization steps.

In some embodiments, the mold is cooled to a freezing temperature of between -10° C. to -190° C. prior to the lyophilization step (iii). In another embodiment, the mold is cooled to a freezing temperature of between -10 to -80° C. for a period of between 0.5 to 24 hours followed by a lyophilization step (iii). In another embodiment, the cooling is carried out for between 0.5-24 hours. In another embodiment, the cooling is carried out for between 0.5-1 hours. In another embodiment, the cooling is carried out for between 1-2 hours. In another embodiment, the cooling is carried out for between 2-5 hours. In another embodiment, the cooling is carried out for between 5-10 hours. In another embodiment, the cooling is carried out for between 10-24 hours. Each possibility represents a separate embodiment of the invention.

In another embodiment, the mold is shaped to afford the desired matrix shape, using a method comprising 3D printing, cast molding or any combination thereof. Each possibility represents a separate embodiment of the invention.

### Method of Preparing an Article of This Invention via Granulates

In one further aspect, this invention provides a method of preparing an article comprising cross-linked collagen, hydroxyapatite, and the method comprises:
(i) providing an acidic solution of collagen, followed by neutralization of the solution wherein the neutralization solution comprises hydroxyapatite;
(ii) concentrating the solution of step (i);
(iii)incubating the composition with a crosslinker and a first solvent;
(iv)washing the incubated composition of step (iv) with a second solvent;
(v) homogenizing, casting and milling the composition to obtain granulates of cross linked collagen;
(vi) wetting the granulates of step (v) by the first or second solvent and carving out to obtain an article comprising cross-linked collagen and hydroxyapatite.

In one emobdiment, this invention provides a method of preparing an article comprising cross-linked collagen, hydroxyapatite and a pharmaceutically active agent, and the method comprises:
(i) providing an acidic solution of collagen, followed by neutralization of the solution wherein the neutralization solution comprises hydroxyapatite;
(ii) concentrating the solution of step (i);
(iii)incubating the composition with a crosslinker, a first solvent and adding a pharmaceutically active agent;
(iv)washing the incubated composition of step (iv) with a second solvent;
(v) homogenizing, casting and milling the composition to obtain granulates of crosslinked collagen;
(vi) wetting the granulates of step (v) by the first or second solvent and carving out to obtain an article comprising cross-linked collagen, hydroxyapatite and a pharmaceutically active agent.

In some embodiments, the matrix obtained by the methods of this invention are milled to form granulates and then wetted by a first or second solvent and carved out to obtain an article of this invention. In another embodiments, the size of the granulates are between 1 to 2000 microns.

In some embodiments, the article prepared according to the methods described hereinabove is sterilized in any method as known in the art (e.g. by ethylene oxide gas (EtO)).

In some embodiments, the collagen used in the methods of this invention within the solution of step “(i)” is selected from the following non-limiting examples including: native collagen, fibrillar collagen, fibrillar atelopeptide collagen, lyophilized collagen, collagen obtained from animal sources, human collagen, recombinant collagen, pepsinized collagen, reconstituted collagen and any combination thereof. In another embodiment, the collagen includes fibrillar collagen reconstituted from monomolecular atelopeptide collagen. In another embodiment, the collagen is atelopeptide fibrillar collagen obtained by reconstituting monomolecular atelopeptide collagen obtained by proteolytic digestion of native collagen. Each possibility represents a separate embodiment of the invention.

In some embodiments, the neutralization solution used in the methods of this invention comprises a base or a buffer. In another embodiment the buffer is selected from a phosphate buffered saline, NaHCO₃/Na₂CO₃ buffer, tris buffer or a tricine buffer or any other buffer that maintains a neutral pH. In another embodiment, the acidic solution comprises HCl, acetic acid, nitric acid, citric acid, sulfuric acid, phosphoric acid or any other acid as known in the art. In another embodiment, the basic solution comprises NaOH, KOH, NaHCO₃, Na₂CO₃, Na₂HPO₄ or any other base as known in the art.

In some embodiments, the term “neutral pH” refers to a range of pHs which resembles the physiological pH in biological body and/or system; and it’s defined as between 6.5-7.5. In some other embodiments, a neutral pH is between 6.5-6.7. In some other embodiments, a neutral pH is between 6.7-6.9. In some other embodiments, a neutral pH is between 6.9-7.1. In some other embodiments, a neutral pH is between 7.1-7.3. In some other embodiments, a neutral pH is between 7.3-7.5. In some other embodiments, a neutral pH is between 7.1-7.2. In some other embodiments, a neutral pH is between 7.2-7.3. In some other embodiments, a neutral pH is between 7.3-7.4. In some other embodiments, a neutral pH is between 7.4-7.5.

In some embodiments, the concentrating step used in the methods of this invention is done by centrifugation. In another embodiment, the centrifugation is carried out at a rate of between 50-20,000 RPM (rounds per minute). In another embodiment, centrifugation is carried out at a rate of between 50-100 RPM. In another embodiment, centrifugation is carried out at a rate of between 100-1,000 RPM. In another embodiment, centrifugation is carried out at a rate of between 1,000-5,000 RPM. In another embodiment, centrifugation is carried out at a rate of between 5,000-10,000 RPM. In another embodiment, centrifugation is carried out at a rate of between 10,000 -20,000 RPM. In another embodiment, centrifugation is carried out for between 1-120 minutes. In another embodiment, centrifugation is carried out for between 1-5 minutes. In another embodiment, centrifugation is carried out for between 5-10 minutes. In another embodiment, centrifugation is carried out for between 10-20 minutes. In another embodiment, centrifugation is carried out for between 20-50 minutes. In another embodiment, centrifugation is carried out for between 50-100 minutes. In another embodiment, centrifugation is carried out for between 100-120 minutes. Each possibility represents a separate embodiment of the invention.

In another embodiment, the lyophilization of step (iii) is carried out for between 1-48 hours. In another embodiment, lyophilization of step (iii) is carried out for between 1-2 hours. In another embodiment, lyophilization of step (iii) is carried out for between 2-5 hours. In another embodiment, lyophilization of step (iii) is carried out for between 5-10 hours. In another embodiment, lyophilization of step (iii) is carried out for between 10-24 hours. In another embodiment, lyophilization of step (iii) is carried out for between 24-48 hours. Each possibility represents a separate embodiment of the invention.

Following the lyophilization, a dried collagen-hydroxyapatite composition of this invention is obtained. The dried composition is incubated with a crosslinker, a first solvent and optionally with a pharmaceutically active agent. The incubated composition is further washed with a second solvent and lyophilized to obtain a matrix comprising cross-linked collagen, hydroxyapatite and optionally a pharmaceutically active agent.

In another embodiment, the first and second solvents are the same or different and selected form any solvent as known in the art. In another embodiment, the solvent is selected from a group comprising: water, ethanol, saline, methanol, phosphate buffer saline or any combination thereof.

In another embodiment, the lyophilization of step (vi) is carried out for between 24 to 72 hrs. In another embodiment, the lyophilization of step (vi) is carried out for between 24 to 36 hrs. In another embodiment, the lyophilization of step (vi) is carried out for between 36 to 48 hrs. In another embodiment, the lyophilization of step (vi) is carried out for between 48 to 72 hrs. Each possibility represents a separate embodiment of the invention.

In some other embodiments, the designed mold and the carving out is planned, designed and/or engineered via computer-aided design (CAD) and/or computer-aided manufacturing (CAM) methods and software as known in the art of the invention. Each possibility represents a separate embodiment of the invention.

In another embodiment, the carving out used within the method of the invention is done by any method as known in the art. In another embodiment, the carving out of the method of the invention is done by a CNC (Computer numerical control) machine, a laser cutting machine, a waterjet cutter, a driller or an abrasive device. Each possibility represents a separate embodiment of the invention.

The following examples are presented in order to more fully illustrate the preferred embodiments of the invention. They should in no way, however, be construed as limiting the broad scope of the invention.

### EXAMPLES

### Example 1

### Preparation of Collagen-Hydroxyapatite Article

30 % w/w hydroxyapatite (CAS® number 12167-74-7, Plasma Biotal Limited (UK)) and 70 % w/w collagen (purified from porcine tendons) components were mixed together during collagen fibrillation. Subsequently, this slurry was cross-linked with sugar (glyceraldehyde). The crosslinked slurry was concentrated by centrifugation and lyophilized. The resulting intermediate dry cake was milled and condensed into round, rectangular or trapezoidal shaped pieces. The compression force which was applied to condense the milled intermediate product varied between 0.005 and 0.2 ton-force. The sterilization of the condensed matter was done by EtO (ethylene oxide gas).

### Example 2

### Bone-Grafting Study of the Crosslinked Collagen-Hydroxyapatite Article in a Rat Model

### Objective

The purpose of this study was to preliminarily evaluate the performance and safety of crosslinked collagen-hydroxyapatite article (prepared as in Example 1) as a bone graft in terms of: 1) regeneration and/or augmentation of bone in a calvaria bone defect model, and 2) foreign body and inflammatory reactions assessed by histological analysis.

### Surgical Procedure

Following anesthesia, an incision was performed posterior to the line between the ears. A full thickness flap was elevated and the calvaria bone was exposed. The soft tissue and periosteum were separated from the bone to the area anterior to the eyes and a defect was created in the calvaria bone without perforating the dura using a 5 mm trephine. Two animals were used in the study. In the first one, a defect of 5 mm in diameter was created, and in the second one, 2 defects of 5 mm each were created in a fuse manner to form an approximately 10 x 5 mm defect. The defects were filled with the crosslinked collagen-hydroxyapatite article and covered with a crosslinked collagen membrane (OSSIX® Plus). Fixation sutures with a resorbable Vicryl 5-0 suture were placed over the OSSIX® Plus and were anchored bilaterally in the temporalis muscles.

### Histological Evaluation

The animals were sacrificed at 9 weeks post-implantation. The calvaria were removed with overlying skin and placed in 10% buffered formalin solution. Following decalcification, the specimens were sectioned and stained with Hematoxylin and eosin (H&E) stain. Evaluation was performed under light microscopy.

### Results

### Clinical Observations

The animals appeared clinically normal throughout the two studies as judged by their weight and food consumption.

### Microscopic Observation

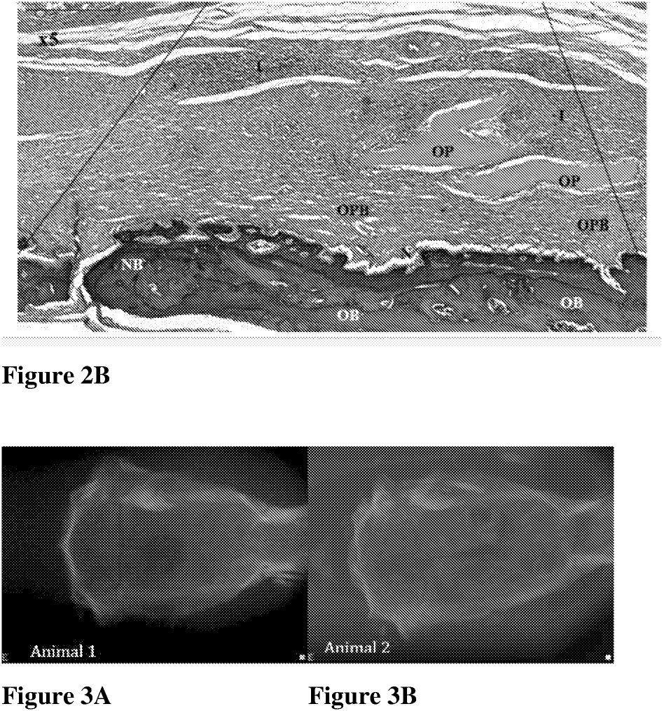

FIGS. 2A-B are microphotographs of the 5 mm defect of animal 1 that was overfilled with the crosslinked collagen-hydroxyapatite article and covered with OSSIX® PLUS. The slides were taken from an off-center location where the defect length was only about 2 mm. The micrographs show a fully closed bone structure beneath the implanted material. Newly formed bone structures can be found adjacent and above the original bone. It should be noted that this animal also showed extensive bleeding during the surgery and suture opening after the surgery not attributed to the device. This resulted in signs of inflammation.

The X-ray opacity in FIG. 3A suggests a closure of the defect but with a bone layer that is thinner than the original bone.

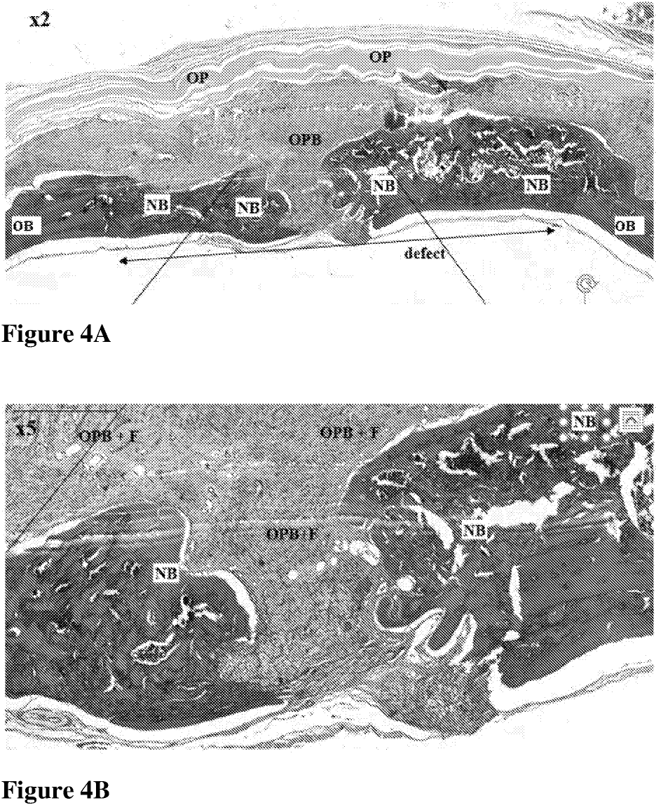

FIGS. 4A-B are overview microphotographs of the 10 mm defect of animal 2 that was overfilled with the crosslinked collagen-hydroxyapatite article and covered with OSSIX® PLUS. The slides were taken from an off-center location where the defect length was about 6.5 mm. The crosslinked collagen-hydroxyapatite article showed evidence of remodeling via soft tissue or via ossification. The micrographs show a 90% closure of the bone defect by newly formed bone. New bone structures can be found adjacent and above the original bone within the crosslinked collagen-hydroxyapatite article. Remodeling to connective tissue is evidenced by the infiltration of fibroblasts above the newly formed bone.

The X-ray opacity (FIG. 3B) of the defect after 9 weeks post implantation suggests an almost complete closure of the defect with a bone layer with thickness similar to the original bone thickness.

### Conclusions

The crosslinked collagen-hydroxyapatite article bone grafting material (in combination with a dental membrane as a barrier membrane (OSSIX® PLUS)) is capable of promoting bone reconstruction and extension above the original bone dimension.

While certain features of the invention have been illustrated and described herein, many modifications, substitutions, changes, and equivalents will now occur to those of ordinary skill in the art. It is, therefore, to be understood that the appended claims are intended to cover all such modifications and changes as fall within the true spirit of the invention.

1. A non-surgical method for treating periodontitis comprising administering an article into a periodontal pocket, wherein the article comprises crosslinked collagen and hydroxyapatite.

2. The method of claim 1, wherein the periodontal pocket is infected.

3. The method of claim 1, wherein the periodontal pocket is not infected.

4. The method of claim 1 , wherein the method leads to reduction of the periodontal pocket depth.

5. The method of claim 1 , where the method does not include administration of an additional pharmaceutically active agent.

6. The method according of claim 1, further comprising administration of at least one pharmaceutically active agent selected from the group consisting of: antibacterial agents, antifungal agents, antiseptic agents, antiinflammatory agents, antibiotic agents, vitamins and vitamers and any combination thereof.

7. The method of claim 6, wherein the pharmaceutically active agent is an antibiotic agent or an antiseptic agent.

8. The method of claim 1 , wherein the method comprises insertion or pushing of the article into the pocket.

9. The method of claim 1, further comprising scaling and root planning, where the scaling and root planning is performed prior to article’s administration.

10. The method of claim 1 , wherein the weight ratio of collagen to hydroxyapatite is between 95:5 and 20:80.

11. The method of claim 10, wherein the weight ratio is 70:30.
